SEQUENCE LISTING (25 sequences)

SEQ ID NO: 1 - DNA, 19 bp, Artificial Sequence
  Porphyromonas gingivalis
tacccatcgtcgccttggt

SEQ ID NO: 2 - DNA, 23 bp, Artificial Sequence
  Porphyromonas gingivalis
cggactaaaaccgcatacacttg

SEQ ID NO: 3 - DNA, 30 bp, Artificial Sequence
  Porphyromonas gingivalis
gctaatgggacgcatgcctatcttacagct

SEQ ID NO: 4 - DNA, 20 bp, Artificial Sequence
  Tannerella forsythia
atcctggctcaggatgaacg

SEQ ID NO: 5 - DNA, 19 bp, Artificial Sequence
  Tannerella forsythia
tacgcatacccatccgcaa

SEQ ID NO: 6 - DNA, 29 bp, Artificial Sequence
  Tannerella forsythia
atgtaacctgcccgcaacagagggataac

SEQ ID NO: 7 - DNA, 22 bp, Artificial Sequence
  Treponema denticola
agagcaagctctcccttaccgt

SEQ ID NO: 8 - DNA, 22 bp, Artificial Sequence
  Treponema denticola
taagggcggcttgaaataatga

SEQ ID NO: 9 - DNA, 25 bp, Artificial Sequence
  Treponema denticola
cagcgttcgttctgagccaggatca

SEQ ID NO: 10 - DNA, 24 bp, Artificial Sequence
  Prevotella intermedia
tgtcggtttactggctatgttctc

SEQ ID NO: 11 - DNA, 23 bp, Artificial Sequence
  Prevotella intermedia
cttgtctgttggccatcttgaag

SEQ ID NO: 12 - DNA, 27 bp, Artificial Sequence
  Prevotella intermedia
tcaaagacgcacgtaccaatccagacc

SEQ ID NO: 13 - DNA, 23 bp, Artificial Sequence
  Fusobacterium nucleatum
cgcagaaggtgaaagtcctgtat

SEQ ID NO: 14 - DNA, 23 bp, Artificial Sequence
  Fusobacterium nucleatum
tggtcctcactgattcacacaga

SEQ ID NO: 15 - DNA, 30 bp, Artificial Sequence
  Fusobacterium nucleatum
actttgctcccaagtaacatggaacacgag

SEQ ID NO: 16 - DNA, 20 bp, Artificial Sequence
  Actinobacillus actinomycetemcomitans
cagcatctgcgatccctgta

SEQ ID NO: 17 - DNA, 23 bp, Artificial Sequence
  Actinobacillus actinomycetemcomitans
tcagccctttgtctttcctaggt

SEQ ID NO: 18 - DNA, 28 bp, Artificial Sequence
  Actinobacillus actinomycetemcomitans
tcgagtattcctcaagcattctcgcacg

SEQ ID NO: 19 - DNA, 25 bp, Artificial Sequence
  Streptococcus mutans
gcctacagctcagagatgctattct

SEQ ID NO: 20 - DNA, 23 bp, Artificial Sequence
  Streptococcus mutans
gccatacaccactcatgaattga

SEQ ID NO: 21 - DNA, 25 bp, Artificial Sequence
  Streptococcus mutans
tggaaatgacggtcgccgttatgaa

SEQ ID NO: 22 - DNA, 23 bp, Artificial Sequence
  Streptococcus sobrinus
ttcaaagccaagaccaagctagt

SEQ ID NO: 23 - DNA, 21 bp, Artificial Sequence
  Streptococcus sobrinus
ccagcctgagattcagcttgt

SEQ ID NO: 24 - DNA, 23 bp, Artificial Sequence
  Streptococcus sobrinus
cctgctccagcgacaaaggcagc

SEQ ID NO: 25 - protein, 12 aa, Homo sapiens
AKRHHGYKRKFH

(19) United States
(12) Patent Application Publication
(10) Pub. No.: US 2017/0211131 A1
(43) Pub. Date: Jul. 27, 2017

(54) METHOD FOR EVALUATING ORAL HEALTH

(71) Applicant: GENERAL BIOLOGICALS CORPORATION, Hsinchu County (TW)

(72) Inventors: Wen-Chi Cheng, Hsinchu County (TW); YI-RU CHANG, Hsinchu County (TW); YU-SHAN WEI, Hsinchu County (TW); Ming-Sun Liu, Hsinchu County (TW); HONG-LIN CHAN, Hsinchu County (TW); Frank Lin, Hsinchu County (TW)

(73) Assignee: GENERAL BIOLOGICALS CORPORATION, Hsinchu County (TW)

(21) Appl. No.: 15/342,105

(22) Filed: Nov. 2, 2016

Related U.S. Application Data
(60) Provisional application No. 62/249,732, filed on Nov. 2, 2015.

(51) Int. Cl. C12Q 1/68; C12Q 1/14; C12Q 1/06

(52) U.S. Cl. CPC C12Q 1/689; C12Q 1/06; C12Q 1/14; C12Q 2600/118; G01N 2333/195; G01N 2333/315; G01N 2800/50; G01N 2800/18

(57) ABSTRACT

The present invention provides a method for evaluating oral health, in particularly for evaluating a risk for suffering from periodontal disease, comprising: (1) providing an oral sample of a subject; (2) detecting at least two indicator microbes in the oral sample; and (3) analyzing amounts of the at least two indicator microbes; wherein when each of the amounts of the at least two indicator microbes is higher than a standard value for an amount of a corresponding indicator microbe, it indicates that the subject has a high risk for suffering from periodontal disease.

This application contains a Sequence Listing in computer readable form. The computer readable form is incorporated herein by reference in its entiriety. This application also claims priority to U.S. Provisional Appl. No. 62/249,732 filed Nov. 2, 2015, which is incorporated herein by reference in its entiriety.

### FIELD OF THE INVENTION

The present invention relates a method for evaluating oral health, specifically for evaluating a risk for suffering from periodontal disease. The method is characterized by analyzing amounts of indicator microbes of periodontal disease in the oral cavity, so as to evaluate the risk for suffering from periodontal disease.

### BACKGROUND OF THE INVENTION

The oral health is an outpost of the human health. When the oral cavity is not healthy, it will affect the life quality in easy-going conditionin and it will induce various systemic diseases in severe condition. Therefore, oral hygiene is very important in maintaining human health. As such, human health is closely related to the healthy condition of the oral cavity. The healthy condition of the oral cavity not only affects aesthetics, pronunciation, chewing and digestion, but also influences the health of the human body.

The physical and psychological health shall be maintained starting from oral care and the teeth are the first concern of a digestive system. Therefore, the teeth care not only reduces occurrence of the periodontal disease, but also promotes the enjoyment of the human life and healthy physical and psychological development of the human body. The periodontal disease can be further divided into two types, one is dental caries and the other is periodontitis, these diseases affect much of oral cavity health.

The dental caries, also known as tooth decay, is one of the most common diseases of the oral cavity, wherein the inorganic part of the tooth is subjected to decalcification, while the organic part of the tooth is subjected to destruction. This chronic disease occurs regardless of sex, age, race, and socioeconomic status. Four factors forming the dental caries are: (1) acid-producing bacteria; (2) host, i.e. teeth and saliva; (3) food; and (4) time. Therefore, the cause of the dental caries can be briefly explained as follows: the “bacterium” capable of causing the dental caries in the oral cavity makes use of the “food” as its nutrients to produce acid after metabolism, the produced acid is then in contact with the teeth for “a long period of time” resulting in the dental caries.

Periodontal disease is a chronic inflammatory disease commonly occured in the world, and it is easily ignored due to no obvious pain. After a long period of time, it will result in bad breath of the oral cavity, swelling and bleeding of the gum, dental plaque, and even tooth removal. The periodontal disease is caused by microbial infection of the peripheral tissues of a tooth, resulting in chronic inflammation. Generally, the microbes combines with protein molecules in the saliva of a host, which are then adhered to the surface of the tooth forming dental plaque, resulting in gingivitis, destruction of gum tissues and eventually alveolar bone loss. Periodontal disease is generally caused by dental biofilms adhered to the surface of the teeth. If the hygiene of the oral cavity is not maintained well, the dental biofilms will accumulate around the gum. The microbes in the dental biofilms will secrete toxins which stimulate the peripheral tissues of the tooth, such as the gum, periodontal membrane and the alveolar bone, rendering periodontitis. If the dental biofilms accumulate, the rim of the gum will show signs of inflammation, resulting in mild periodontitis (gingivitis). When the immune system is compromised, body tissues recover poorly, periodontal tissues, including the gum, the periodontal membrane and the alveolar bone, will be subjected to destruction and the body condition will deteriorate, thereby forming severe periodontitis (periodontitis).

In 1998, Socransky et. al. investigated 185 people, some in healthy condition and the others suffering from periodontitis, and analyzed 13,261 samples of the dental plaque collected under the gum, they divided microbes in the oral cavity into five varieties according to the clinical course and their effects on periodontal disease. These microbes are classified into five groups, red, orange, yellow, green and violet according to the degree of their effects on the periodontal disease. Later study includes blue as the sixth group covering the remaining microbes in the oral cavity. It has been found that three most important varieties of microbes are porphyromonas gingivalis (Pg), Tannerella forsythia (Tf) and Treponema denticola (Td), which constitute the group of microbes most related to the formation of the periodontitis, being entitled as the “red complex.”

P. gingivalis is a gram negative anaerobic bacterium, which is in black rod-shape, capable of adhering to other bacteria, epithelial cells and extracellular matrix proteins, secreting toxins, hemolysin to destruct the host tissues, and evading defensive ability of the host, thereby invading the host cells via altering the cytoskeletal structure. T. forsythia is also a gram negative anaerobic bacterium, capable of secreting toxin-causing factors to provoke extreme immune responses, separating the periodontal ligaments and dissolving the alveolar bone as a result. Its pathogenicity adheres to both Pg and Td and they work together.

The health of the gum tissues may be recovered by cleaning the dental plaque from patients suffering from mild gingivitis. However, the condition of inflammation continues for patients suffering from more serve gingivitis, separating the gum and the root portion of the tooth to form a pouch which allows food residues, dental plaques and dental calculus to accumulate, thereby causing the periodontal disease. The course of the periodontal disease is divided into four stages, i.e. gingivitis, mild periodontitis, moderate periodontitis and serve periodontitis. Not all the gingivitis will transform into the periodontitis. Therefore, daily oral health care and routine monitor of the oral cavity become quite important.

In addition, a high percentage of the adult population in Taiwan suffer from periodontitis, and they are often unconscious of disease due to mild symptoms of associated with periodontitis. According to the statistics published by the Taiwan Academy of Periodontology (Taiwan Academy of Periodontology), less than 40% of periodontitis patients sought for medical treatment. During the course of periodontitis, the destruction is slow and accumulation of dental plaques is continuous, which often induce tooth loss in untreated elderly patients suffering from periodontal disease. The major solution for tooth loss is “dental implant.” During the course of dental implant, an artificial tooth root made of titanium is implanted in the alveolar bone as a replacement of the original tooth root of the tooth, after three to six months the implant and the bone are closely integrated and a denture is made on the titanium tooth root to restore appearance and chewing function. Nowadays, dental implant is very common because of advanced technology and sophisticated materials. After dental implantation, the appearance and function of the tooth can be very close to that of the original tooth.

Many people, after dental implantation, ignore the importance of oral hygiene because they mistakenly believe that their dentures, unlike natural teeth, are immune to periodontal disease because the artificial tooth root is made of metal, resulting in “peri-implant periodontitis.” Symptoms suffered by peri-implant periodontitis patients are inflammation around their alveolar bones, gums and other parts, including soft tissue hyperplasia, purulent, bleeding, redness, fistula, pain and other conditions. The implant may begin to shake, once it is shaken there is no treatment and it must be removed. Moreover, the material used to make artificial tooth root, unlike natural tooth root, does not contain sensitive sensory nerves and other structures, the body is unable to sense damages caused to the surrounding tissue, resulting in even more serious peri-implant periodontitis and early exposure of the artificial tooth root. Statistically, about 25 to 50% of patients with dental implants suffer from peri-implant periodontitis.

Currently, the risk of oral health endangered by periodontal disease causing damages to oral health such as dental caries or periodontitis can only be reduced through daily self-maintenance of oral hygiene. Patients who usually seek for medical treatment after they have a toothache miss the opportunity to treat the disease in early stages.

Therefore, it would be of great help, through analysis, research and development of oral flora compositions, to establish a set of optimal indicators for periodontal disease diagnosis, which would serve as effective bases for daily oral health care, monitoring of periodontal disease, and even antibiotic treatment for severe periodontal disease patients, and follow-up and investigation of dental implants after implantation.

### SUMMARY OF THE INVENTION

The present invention relates to an evaluation of oral health, specifically an evaluation of a risk for suffering from periodontal disease, which comprises: (1) providing an oral sample from a subject; (2) detecting at least two indicator microbes in the oral sample; and (3) analyzing an amount of each of the at least two indicator microbes; wherein when the amount of each of the least two indicator microbes is high than a standard value for an amount of a corresponding indicator microbe, it indicates that the subject has a high risk for suffering from periodontal disease.

### BRIEF DESCRIPTION OF THE DRAWINGS

FIG. 1 shows the standard curve used for calculating the amount of Porphyromonas gingivalis.

FIG. 2 shows the standard curve used for calculating the amount of Tannerella forsythia.

FIG. 3 shows the standard curve used for calculating the amount of Prevotella intermedia.

FIG. 4 shows the standard curve used for calculating the amount of Fusobacterium nucleatuni.

FIG. 5 shows the standard curve used for calculating the amount of Actinobacillus actinomycetemcomitans.

FIG. 6 shows the results of the bacteria counts of Actinobacillus actinomycetemcomitans (Aa), Prevotella intermedia (Pi), Fusobacterium nucleatuni (Fn), Porphyromonas gingivalis (Pg), Treponema denticola (Td), and Tannerella forsythia (Tf) of a healthy subject group and a periodontitis patient group detected by using real-time PCR molecular detection method.

FIG. 7 shows the detection results of the amounts of Actinobacillus actinomycetetemcomitans (Aa), Prevotella intermedia (Pi), Fusobacterium nucleatum (Fn), Porphyromonas gingivalis (Pg), Treponema denticola (Td), and Tannerella forsythia (Tf) before and after the periodontal disease patients receive the treatment for periodontitis.

FIG. 8 shows the establishment of a handling method used before the sample identification procedure is performed by the MALDI Biotyper.

FIG. 9 shows images of serial dilution of the oral samples. (A) diluted 20-fold; (B) diluted 200-fold; (C) diluted 1000-fold; and (D) diluted 2000-fold are to select one single colony which is the best condition applicable to MALDI Biotyper.

FIG. 10 shows the distribution of the periodontitis indicator microbial strains in healthy subjects and in periodontitis patients. In each bar diagram, when each set of data is arranged in the order from the left, the first three groups are saliva samples of healthy subjects, periodontitis patients under the age of 60, and periodontitis patients over the age of 60, respectively; the last three groups are subgingival samples of healthy subjects, periodontitis patients under the age of 60, and periodontitis patients over the age of 60, respectively. (A) Microflora distribution rate of Actinobacillus actinomycetetemcomitans, (B) Microflora distribution rate of Fusobacterium nucleatum, (C) microflora distribution rate of Porphyromonas gingivalis.

FIG. 11 shows the distribution of the amounts of microbes in the oral saliva samples of 6 tested healthy subjects before the use of P-113 mouthwash. (A) A comparison of the amount of Porphyromonas gingivalis; (Pg) before the use of P-113 mouthwash and after the the use of P-113 mouthwash, the amounts of bacteria in all 6 tested subjects decrease significantly; (B) The amount of Tannerella forsythia (TF) also shows a trend toward significantly decrease before and after the use.

FIG. 12 shows the average microbial inhibition rate of Porphyromonas gingivalis (Pg) and Tannerella forsythia (TF) in 6 tested healthy subjects.

FIG. 13 shows the amount of each micribial strain in the mixed saliva samples of 15 tested healthy subjects before and after the use of P-113 mouthwash.

FIG. 14 is a structural diagram of the oral health detection and evaluation system.

FIG. 15 is the oral health evaluating table of the present invention.

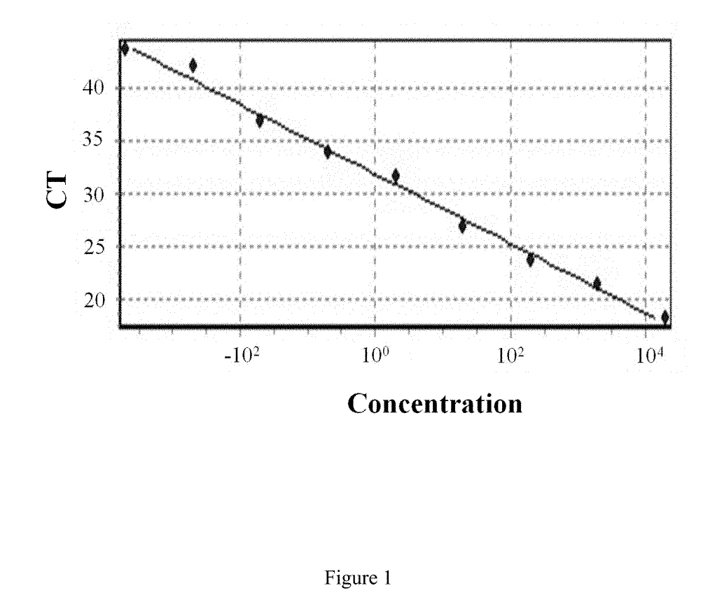

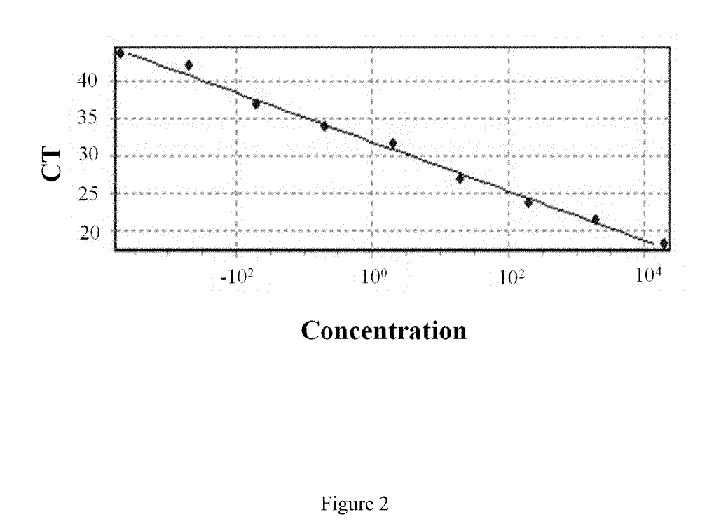

### DETAILED DESCRIPTION OF THE INVENTION

The present invention aims to establish an optimal standard applicable to a periodontal disease diagnostic process that is used for monitoring the oral health and the clinical course of periodontal disease, evaluating a treatment effect in periodontal patients, and tracking and investigating patients with dental implants after the dental implantation. The present invention contributes to oral health care and decreases a risk for suffering from periodontal disease.

A basic periodontal disease diagnostic service process of the present invention comprises: (1) collecting an oral sample, wherein the oral sample is saliva obtained by a subject using a saliva collecting tube or a subgingival sample collected via an assistance of a dentist; (2) extracting DNA from the oral sample by a medical laboratory scientist in a laboratory; (3) detecting indicator microbes of periodontal disease and assaying amounts of indicator microbes of periodontal disease by using a molecular diagnostic method; (4) making an assay report of the periodontal bacteria in the oral cavity based on detecting results; and (5) sending the assay report of the periodontal bacteria in the oral cavity to the dentist or the subject.

The present invention provides a method for evaluating a risk for suffering from periodontal disease, comprising: (1) providing an oral sample from a subject; (2) detecting at least two indicator microbes in the oral sample; and (3) analyzing amounts of the at least two indicator microbes, wherein when each of the amounts of the at least two indicator microbes is higher than a standard value for an amount of a corresponding indicator microbe, it indicates that the subject has a high risk for suffering from periodontal disease.

In one embodiment, the periodontal disease comprises periodontosis and dental caries. In a preferred embodiment, the periodontosis comprises periodontitis and gingivitis.

The term “subject” used herein is an animal. In a preferred embodiment, the subject is a mammal. In a more preferred embodiment, the subject is a human.

In another embodiment, the oral sample is an oral mucosa, a subgingival mucosa or a saliva.

When the method of the present invention is used for evaluating a risk for suffering from periodontosis, the at least two indicator microbes comprise periodontosis-related indicator microbes, wherein the periodontosis-related indicator microbes comprise Porphyromonas gingivalis, Tannerella forsythia, Treponema denticola, Prevotella intermedia, Fusobacterium nucleatum, Actinobacillus actinomycetemcomitans or a combination thereof. In one embodiment, the at least two indicator microbes comprise Porphyromonas gingivalis, Tannerella forsythia, Treponema denticola, Prevotella intermedia, Fusobacterium nucleatum, and Actinobacillus actinomycetemcomitans.

In another embodiment, the range of standard values for the amounts of the corresponding indicator microbes comprise the standard value for the amount of the Porphyromonas gingivalis ranging from 300-3000, the standard value for the amount of the Tannerella forsythia ranging from 100-2000, the standard value for the amount of the Treponema denticola ranging from 100-2000, the standard value for the amount of the Prevotella intermedia ranging from 20-1000, the standard value for the amount of the Fusobacterium nucleatum ranging from 8000-80000, and the standard value for the amount of the Actinobacillus actinomycetemcomitans ranging from 20-200. In a preferred embodiment, the standard values for the amounts of the corresponding indicator microbes comprise the standard value for the amount of the Porphyromonas gingivalis which is 1000, the standard value for the amount of the Tannerella forsythia which is 600, the standard value for the amount of the Treponema denticola which is 800, the standard value of the amount of the Prevotella intermedia which is 100, the standard value for the amount of the Fusobacterium nucleatum which is 10000, and the standard value for the amount of the Actinobacillus actinomycetemcomitans which is 100. Therefore, when the amounts of some periodontosis-related disease indicator microbes in the oral sample are higher than the corresponding standard values thereof, for example, the amount of the Porphyromonas gingivalis in the oral sample is higher than 1000, which may indicate that the subject has a high risk for suffering from periodontosis.

When the method of the present invention is used for evaluating a risk for suffering from dental caries, the at least two indicator microbes comprise dental caries-related indicator microbes, wherein the dental caries-related indicator microbes comprise Streptococcus mutans and Streptococcus sobrinus. In one embodiment, the at least two indicator microbes comprise Streptococcus mutans and Streptococcus sobrinus. In a preferred embodiment, the standard values for the amounts of corresponding indicator micbrobes comprise the standard value for the amount of the Streptococcus mutans ranging from 300-3000 and the standard value for the amount of the Streptococcus sobrinus ranging from 300-3000. Therefore, when the amounts of some dental-related caries indicator microbes in the oral sample are higher than the corresponding standard values thereof, for example, the amount of the Streptococcus mutans in the oral sample is higher than 1000, which may indicate that the subject has a high risk for suffering from dental caries.

In another embodiment, the method for detecting the at least two indicator microbes in the oral sample comprises a PCR, a real-time PCR, a multiplex real-time PCR, a digital PCR, a droplet digital PCR (ddPCR) and a PCR array.

In one embodiment, the method for detecting the at least two indicator microbes in the oral sample comprises MALDI Biotyper detecting method. The MALDI Biotyper identifies a microorganism by measuring a unique molecular fingerprint of an organism using the matrix assisted laser desorption ionization-time of flight mass spectrometry (MALDI-TOF). In particular, the MALDI Biotyper is able to measure and find highly abundant proteins in all microorganisms.

In one embodiment, the present invention further comprises a treating step, comprising treating the subject who is determined to have a high risk for suffering from periodontal disease.

The term “treating” of any disease or disorder refers to ameliorating the disease or disorder (i.e., arresting the progression of the disease or reducing the manifestation, extent or severity of at least one of the clinical symptoms thereof). In a preferred embodiment, the treating step comprises administering to the subject a mouthwash containing an antimicrobial peptide. In a more preferred embodiment, the route of the administering to the subject the mouthwash is an oral administration.

In another embodiment, the antimicrobial peptide is a P-113 peptide consisting of 12 amino acids of histatin-5. The sequence of the P-113 peptide can be referred to U.S. Pat. Nos. 5,631,228, 5,646,119, 5,885,965, 5,912,230 and 15,175,011. The content of the above-mentioned patents teach a definition of an amino acid sequence of the histatin family and an amino acid sequence of P-113.

Therefore, the P-113 peptide is 12 peptides, and the amino acid sequence of P-113 peptide comprises SEQ ID NO: 25, the amino acid sequence thereof is AKRHHGYKRKFH. In a preferred embodiment, the P-113 peptide is a modified P-113 peptide fragment. In a more preferred embodiment, the modification comprises repeating sequences of the P-113 peptide, protein processing, glycosylation, carboxy terminal amidation or amino acid isomerization.

The present invention also provides a service process for oral health diagnosis, which comprises: (a) collecting an oral sample from a subject; (b) detecting at least two indicator microbes in the oral sample and analyzing amounts of the indicator microbes; (c) comparing each of the amount of the at least two indicator microbes to a standard value for an amount of a corresponding indicator microbe; and (d) making an oral health test report based on comparison results of step (c).

In one embodiment, the oral health diagnosis is a periodontal disease diagnosis. In a preferred embodiment, the oral health diagnosis is a diagnosis of periodontitis or a diagnosis of dental caries (tooth decay).

In another embodiment, the oral sample is an oral mucosa, a subgingival mucosa or a saliva. The saliva can be collected by a subject using a saliva collecting tube, and the subgingival sample of the subject can be collected through an assistance of a dentist.

In one embodiment, the method for detecting the at least two indicator microbes in the oral sample is performed by extracting the bacterial DNA of the oral sample for molecular diagnostic assay. In a preferred embodiment, the molecular diagnostic assay comprises a PCR, a real-time PCR, a multiplex real-time PCR, a digital PCR, a droplet digital PCR (ddPCR) and PCR array.

In one embodiment, the method for detecting the at least two indicator microbes in the oral sample comprises MALDI Biotyper detecting method.

When the service of the oral health diagnosis is a diagnosis of dental caries (tooth decay), the at least two indicator microbes comprise

Streptococcus mutans and Streptococcus sobrinus. In a preferred embodiment, the standard values for the amounts of the corresponding indicator microbes comprise the standard value for the amount of the Streptococcus mutans ranging from 300-3000 and the standard value for the amount of the Streptococcus sobrinus ranging from 300-3000.

In one embodiment, a primer for detecting the Streptococcus mutans comprises SEQ ID NO: 19 and SEQ ID NO: 20. In a preferred embodiment, a fluorescent probe that detects the Streptococcus mutans comprises SEQ ID NO: 21. In another embodiment, a primer for detecting the Streptococcus sobrinus comprises SEQ ID NO: 22 and SEQ ID NO: 23. In a preferred embodiment, a fluorescent probe for detecting the Streptococcus mutans comprises SEQ ID NO: 24.

When the service of the oral health diagnosis is a diagnosis of periodontal disease, the at least two indicator microbes comprise Porphyromonas gingivalis, Tannerella forsythia, Treponema denticola, Prevotella intermedia, Fusobacterium nucleatum, and Actinobacillus actinomycetemcomitans.

In one embodiment, the standard values for the amounts of the corresponding indicator microbes comprise the standard value for the amount of the Porphyromonas gingivalis ranging from 300-3000, the standard value for the amount of the Tannerella forsythia ranging from 100-2000, the standard value for the amount of the Treponema denticola ranging from 100-2000, the standard value for the amount of the Prevotella intermedia ranging from 20-1000, the standard value for the amount of the Fusobacterium nucleatum ranging from 8000-80000, and the standard value for the amount of the Actinobacillus actinomycetemcomitans ranging from 20-200. In a preferred embodiment, the standard values for the amounts of the corresponding indicator microbes comprise the standard value for the amount of the Porphyromonas gingivalis which is 1000, the standard value for the amount of the Tannerella forsythia which is 600, the standard value for the amount of the Treponema denticola which is 800, the standard value for the amount of the Prevotella intermedia which is 100, the standard value for the amount of the Fusobacterium nucleatum which is 10000, and the standard value for the amount of the Actinobacillus actinomycetemcomitans which is 100. Therefore, when the amount of the Porphyromonas gingivalis in the oral sample is higher than the standard value of 1000 and the other indicator microbes also exceed the standard values, it indicates that the subject has a high risk for suffering from periodontitis.

In one embodiment, a primer for detecting Porphyromonas gingivalis comprises SEQ ID NO: 1 and SEQ ID NO: 2. In a preferred embodiment, a fluorescent probe for detecting Porphyromonas gingivalis comprises SEQ ID NO: 3.

In another embodiment, a primer for detecting Tannerella forsythia comprises SEQ ID NO: 4 and SEQ ID NO: 5. In a preferred embodiment, a fluorescent probe for detecting Tannerella forsythia comprises SEQ ID NO: 6.

In one embodiment, a primer for detecting Treponema denticola comprises SEQ ID NO: 7 and SEQ ID NO: 8. In a preferred embodiment, a fluorescent probe for detecting the Treponema denticola comprises SEQ ID NO: 9.

In another embodiment, a primer for detecting Prevotella intermedia comprises SEQ ID NO: 10 and SEQ ID NO: 11. In a preferred embodiment, a fluorescent probe for detecting Prevotella intermedia comprises SEQ ID NO: 12.

In one embodiment, a primer for detecting Fusobacterium nucleatum comprises SEQ ID NO: 13 and SEQ ID NO: 14. In a preferred embodiment, a fluorescent probe for detecting Fusobacterium nucleatum comprises SEQ ID NO: 15.

In another embodiment, a primer for detecting Actinobacillus actinomycetetemcomitans comprises SEQ ID NO: 16 and SEQ ID NO: 17. In a preferred embodiment, a fluorescent probe for detecting Actinobacillus actinomycetetemcomitans comprises SEQ ID NO: 18.

There are currently at least 700 identified bacteria in the oral cavity, and a variety of bacteria are distributed in different ratios in the oral cavity between a healthy subject and a periodontal patient. Therefore, the service process of the present invention can also use molecular diagnostic techniques (for example MALDI Biotyper) to identify other oral microbes to assess the level of oral health or the level of a risk for suffering from periodontal disease.

In one embodiment, the oral health assay report comprises comparison results of the indicator microbes in the oral sample and an analysis and description of the comparison results. In a preferred embodiment, the analysis and description of the comparison results is a subject's level of risk for suffering from periodontitis or dental caries.

In one embodiment, the oral health assay report is sent to the dentist or to the subject. In a preferred embodiment, the method for sending the report comprises an ordinary mail or an e-mail. In another embodiment, the oral health assay report is stored in a cloud storage device. The subject can use a computer device to connect to the cloud storage device to view the oral health assay report. In addition, the cloud storage device connects to an on-line commentary service which provides explanations for the oral health assay report and replies to questions raised by the subject.

The service process of the present invention further comprises a treating step after step (d), which comprises treating the subject when the subject is determined to have a high risk for suffering from periodontal disease or dental caries. In a preferred embodiment, the treating step comprises administering to the subject a mouthwash containing an antimicrobial peptide. In a more preferred embodiment, the antimicrobial peptide is a P-113 peptide.

The present invention further provides a use of a composition for preparing a pharmaceutical composition for treating periodontal disease, wherein the composition comprises a P-113 peptide, wherein the sequence of the P-113 peptide comprises SEQ ID NO: 25.

In one embodiment, the periodontal disease comprises periodontitis and dental caries. In a preferred embodiment, the periodontitis comprises periodontitis and gingivitis.

In one embodiment, a form of the pharmaceutical composition is a mouthwash. In a preferred embodiment, a route of an administration of the pharmaceutical composition is an oral administration.

In another embodiment, the P-113 peptide is a modified P-113 peptide. In a more preferred embodiment, the modification comprises repeating sequences of the P-113 peptide fragment, protein processing, glycosylation, carboxy terminal amidation or amino acid isomerization.

The term “peptide” as used herein typically refers to a peptide shorter in length. Therefore, peptides, oligopeptides, dimers, multimers and the like are within the scope as defined. The definition intends to cover full-length proteins and fragments thereof. The term “polypeptide” and “protein” also includes post-expression modification of polypeptides and proteins, for example, glycosylation, acetylation, phosphroylation and the like. For purposes of the present invention, “polypeptide” may include “modification” of a native sequence, such as deletion, insertion, substitution (the nature could be conservative or include the following substitution: any one of the 20 amino acids normally found in human proteins, or any other naturally or non-naturally occurring amino acids or atypical amino acids) and chemical modification (insertion of or substitution with mimetic peptides). These modifications could be deliberate or site-directed mutagenesis, or by chemically modifying amino acid to delete or connect chemical moieties, or may be accidental, for example, due to mutation induced by protein-generating hosts or due to mistakes caused by PCR amplifications.

In one embodiment, the P-113 peptide treats the periodontitis by inhibiting a bacterium in an oral cavity. In a preferred embodiment, the bacterium comprises Porphyromonas gingivalis, Tannerella forsythia, Treponema denticola, Prevotella intermedia, Fusobacterium nucleatuni, and Actinobacillus actinomycetenicomitans.

In one embodiment, the P-113 peptide treats dental caries by inhibiting a bacterium in an oral cavity. In a preferred embodiment, the bacterium comprises Streptococcus mutans and Streptococcus sobrinus.

The term “inhibiting a bacterium in an oral cavity” as used herein includes killing bacteria, eliminating bacteria, disinfecting, suppressing bacteria, anti-mildew or anti-mitotic, etc.

In another embodiment, the P-113 peptide reduces the occurrence of periodontitis by increasing Streptococcus salivarius to balance microflora in an oral cavity.

In addition, the present invention provides a method for treating periodontal disease comprising administering to a subject an effective amount of a pharmaceutical composition, wherein the pharmaceutical composition comprises a P-113 peptide, wherein the sequence of the P-113 peptide comprises SEQ ID NO: 25.

In one embodiment, the subject is a subject suffering from periodontal disease.

In one embodiment, the periodontal disease comprises periodontosis and dental caries. In a preferred embodiment, the periodontosis comprises periodontitis and gingivitis.

The term “an effective amount” used herein is a therapeutic dose which can prevent, decrease, stop or reverse a symptom developed in a subject under specific conditions, or partially, completely alleviates symptoms already exist under specific conditions when the subject begins receiving the treatment.

The pharmaceutical composition further comprises a pharmaceutically acceptable carrier. The term “a pharmaceutically acceptable carrier” as used herein is determined by the specific combination and the specific method the composition is administered. The term “carrier” as used herein includes but is not limited to any and all solvents, dispersing media, vehicles, coatings, diluents, antibacterial and antifungal agents, penetration and absorption delaying agents, buffers, carrier solutions, suspension fluids, colloidal gels, etc. These media and reagents used as active ingredients of the pharmaceutical composition are well-known in the art. If a conventional medium or reagent is incompatible with any of the active ingredients, care must be taken when it is used in a composition for treatment purposes. Complementary active ingredients may also be incorporated into the composition. The term “pharmaceutically acceptable” as used herein refers to molecular entities and compositions administered to a subject without causing any allergic reactions or similar negative effects.

The form of the pharmaceutical composition can be prepared as aerosols, tablets, pills, capsules, sterile powders, suppositories, lotions, creams, ointments, pastes, gels, hydrogels, sustained delivery devices, or other formulations which may be used for drug delivery. In a preferred embodiment, a form of the pharmaceutical composition is a mouthwash. In a more preferred embodiment, a route of an administration of the pharmaceutical composition is an oral administration.

In one embodiment, the P-113 peptide treats the periodontosis by inhibiting a bacterium in an oral cavity. In a preferred embodiment, the bacterium comprises Porphyromonas gingivalis, Tannerella forsythia, Treponema denticola, Prevotella intermedia, Fusobacterium nucleatum, and Actinobacillus actinomycetemcomitans. In another embodiment, the bacterium comprises Streptococcus pneumonia, Streptococcus parasanguinis, Veillonella parvula, Veillonella dispar, Streptococcus oxalis, Veillonella atypical, Streptococcus peroris, Prevotella melaninogenica, Streptococcus mitis and Actinomyces oris.

In addition, the P-113 peptide prevents an occurrence of periodontosis by increasing Streptococcus salivarius.

In another embodiment, the P-113 peptide treats dental caries by inhibiting a bacterium in an oral cavity. In a preferred embodiment, the bacterium comprises Streptococcus mutans and Streptococcus sobrinus.

The present invention provides an oral health detection and evaluation system comprising: (1) an oral microflora detecting device which is used for detecting a distribution of the oral microflora in an oral sample and for generating a microflora distribution data; (2) an oral microflora colony database which stores an oral microflora colony comparison table; and (3) an oral health evaluating device connected to the oral microflora detecting device and the oral microflora colony database, which is used for comparing the microflora distribution data and the oral microflora colony comparison table and then generating an oral health evaluating table based on the comparison results.

In a particular embodiment, the oral health detection and evaluation system further comprises a sampling device connected to the oral microflora detecting device, which is used for collecting the oral sample. In a preferred embodiment, the oral sample is an oral mucosa, a subgingival mucosa or a saliva.

In another embodiment, the oral microflora detecting device comprises a PCR device and a microflora analyzer. The PCR device uses a specific primer of a specific microbe to perform polymerase chain reaction of DNA in an oral sample, amplifies an amount of the DNA to determine quantitatively the DNA concentration of the specific microbe, and then to estimate an amount of the specific microbe. In a preferred embodiment, the microflora analyzer is a matrix assisted laser desorption ionization-Time of flight mass spectrometry (MALDI-TOF) which can be used for analyzing and identification of microbes.

In one embodiment, the microflora distribution data comprise a set of data for identifying the varieties of microbes and a set of data for the amounts of microflora colonies. Therefore, the microflora distribution data can show data of periodontal disease-related indicator microbes and the amounts thereof.

In another specific embodiment, the oral microflora colony database comprises an oral health control group data, a microbial characteristic data, a periodontosis-related data, and a dental caries-related data. The oral health control group data comprises a set of data for the distribution and amounts of microbes in a healthy oral cavity, which are used for comparing with and analyzing microbial colonies in a to-be-tested oral sample. The microbial characteristic data comprises a set of data for the characteristics of each variety of oral microbes and periodontal diseases possibly to be induced. The periodontosis-related data comprises the risk and the progression determination of periodontosis and related symptoms/diseases thereof. The dental caries-related data comprises the risk and the progression determination of dental caries and related symptoms/diseases thereof. The above-mentioned data are stored in the oral microflora colony database for cross-comparison analysis.

In one embodiment, the oral microflora colony comparison table comprises a comparison table of the varieties and standard values for the amounts of periodontosis-related indicator microbes and a comparison table for periodontosis-related diseases. In a preferred embodiment, the comparison table of the varieties and standard values for the amounts of periodontosis-related indicator microbes comprises a standard value for the amount of Porphyromonas gingivalis ranging from 300-3000, a standard value for the amount of Tannerella forsythia ranging from 100-2000, a standard value for the amount of Treponema denticola ranging from 100-2000, a standard value for the amount of Prevotella intermedia ranging from 20-1000, a standard value for the amount of Fusobacterium nucleatum ranging from 8000-80000, and a standard value for the amount of Actinobacillus actinomycetemcomitans ranging from 20-200. In a preferred embodiment, the comparison table of the varieties and standard values for the amounts of periodontitis-related indicator microbes comprises the standard value for the amount of the Porphyromonas gingivalis which is 1000, the standard value for the amount of the Tannerella forsythia which is 600, the standard value for the amount of the Treponema denticola which is 800, the standard value for the amount of the Prevotella intermedia which is 100, the standard value for the amount of the Fusobacterium nucleatum which is 10000, and the standard value for the amount of the Actinobacillus actinomycetemcomitans which is 100.

In another embodiment, the oral microflora colony comparison table comprises a comparison table of the varieties and standard values for the amounts of dental caries-related indicator microbes and a comparison table for dental caries-related diseases. In a preferred embodiment, the comparison table of the varieties and standard values for the amounts of dental caries-related indicator microbes comprises a standard value for the amount of Streptococcus mutans ranging from 300-3000 and a standard value for the amount of Streptococcus sobrinus ranging from 300-3000.

In one embodiment, the oral health evaluating table comprises an index table for comparison of the amounts of periodontosis-related indicator microbes and an evaluating table for periodontal disease risk index. Therefore, by comparing the microflora distribution data of the oral sample and the oral microflora colony comparison table, the differences between the varieties and the amounts of periodontosis-related indicator microbes in the oral sample and the varieties and standard values for the amounts of periodontosis-related indicator microbes are identified to generate the index table for comparison of the amounts of periodontosis-related indicator microbes. Then, a risk for suffering from periodontosis-related disease is further determined according to the identified differences resulting from the comparison; i.e., an evaluating table for periodontal disease risk index is therefore generated.

In one embodiment, the oral health evaluating table comprises an index table for comparison of the amounts of dental caries-related indicator microbes and an evaluating table for dental caries-related disease risk index. Therefore, by comparing the microflora distribution data of the oral sample and the oral colony comparison table, the differences between the varieties and the amounts of the microbes in the oral sample and the varieties and standard values for the amounts of dental caries-related indicator microbes are identified to generate the index table for comparison of the amounts of dental caries-related indicator microbes. Then, a risk for suffering from dental caries-related diseases is further determined according to the identified differences resulting from the comparison; i.e., the evaluating table for dental caries-related disease risk index is therefore generated.

The term “microbe”, “microbial colony” or “strain” as used herein includes, but is not limited to, a bacterium and a fungus. In a preferred embodiment, the periodontosis-realted indicator microbes include, but are not limited to, Porphyromonas gingivalis, Tannerella forsythia, Treponema denticola, Prevotella intermedia, Fusobacterium nucleatuni, and Actinobacillus actinongcetenicomitans.

In one embodiment, the dental caries-related indicator microbes include, but are not limited to, Streptococcus mutans and Streptococcus sobrinus.

In one embodiment, the periodontal disease comprises periodontosis and dental caries. In a preferred embodiment, the periodontosis comprises periodontitis and gingivitis.

In one embodiment, the oral health evaluating device further comprises an analysis result storage element that can be used to store the oral health evaluating table. In a preferred embodiment, the oral health evaluating device further comprises a result output device, connected to the analysis result storage element, for outputting the oral health evaluating table. In a more preferred embodiment, the result output device is a network transmission device, a telefax facsimile, a display, a printer, a computer system, an electronic application device or a memory storage device.

In another embodiment, the analysis result storage element is further connected to a cloud server. In a preferred embodiment, the cloud server is connected to a personal computer device. In a more preferred embodiment, the personal computer device includes, but is not limited to, a smartphone, a desktop computer, a notebook computer, a tablet computer, and a smart wearable device.

In one embodiment, the personal computer device is connected to the cloud server through a user interface. In a preferred embodiment, the user interface is displayed on a display of the personal computer device. The person to be tested can receive and view the oral health evaluating table of the analysis result storage element through the user interface to understand personal oral health condition. In addition, the user interface further provides an online commentary service that explains the content of the oral health evaluating table and provides advices on oral care or treatment.

Therefore, the oral health detection and evaluation system can provide a diagnostic service for a risk of suffering from periodontosis or dental caries.

When a tested person is determined to have a high risk for suffering from periodontosis or have been suffering from periodontosis according to the oral health evaluating table, the tested person can further receive a periodontal treatment course, for example, using a mouthwash containing an antimicrobial peptide P-113 to effectively balance and inhibit oral pathogens in order to achieve oral health effects. The antimicrobial peptide P-113, derived from histatin-5, has been reported to have antimicrobial efficacy in many studies and literature (referred as U.S. Pat. Nos. 5,631,228, 5,646,119, 5,885,965 and 5,912,230) and can be used to inhibit microflora in the oral cavity.

Therefore, after the tested person receiving the course of treatment, the person uses the oral health detection and evaluation system of the present invention to constantly track changes of the microflora in the oral cavity in order to monitor the treating effect and establish personalized oral health indicators.

### EXAMPLES

The embodiment of the present invention could be implemented with different content and is not limited to the examples described in the following text. The following examples are merely representative of various aspects and features of the present invention.

1. Preparation of Periodontosis-Related Microflora and Molecular Detection Methods

1.1 Design of PCR Primers for Microflora Detection

The molecular diagnostic and detection method includes, but is not limited to, polymerase chain reaction (PCR) analyzed by general electrophoresis, polymerase chain reaction (PCR) analyzed by capillary electrophoresis, Real-time polymerase chain reaction, multiplex real-time PCR, digital PCR, droplet digital PCR (ddPCR), PCR Array or other methods.

Periodontosis-related microflora to be detected were as follows: (1) Pg: Porphyromonas gingivalis; (2) Tf: Tannerella forsythia; (3) Td: Treponema denticola; (4) Pi: Prevotella intermedia; (5) Fn: Fusobacterium nucleatuni; and (6) Aa: Actinobacillus actinomycetenicomitans. The design of Real-time PCR primers for periodontosis-related bacteria is shown in Table 1 which also provides the sequence of fluorescent probes: 5′-end containing FAM (6-carboxyfluorescein) and 3′-end containing TAN′IRA dye, i.e., the quencher dye (6-carboxytetramethylrhodamine).

TABLE 1
Design of Real-time PCR primers and fluorescent probes for
periodontitis-related bacteria

| | | | SEQ | |
|---|---|---|---|---|
| | | Amplicon | ID | Target |
| Culture | Primer sequence | size (bp) | NO | gene |
| Pg | Pg-F 5′-TACCCATCGTCGCCTTGGT-3′ | 126 | 1 | 16S |
| | Pg-R 5′-CGGACTAAAACCGCATACACTTG-3′ | | 2 | rRNA |
| | Fluorescent probe | | | |
| | Pg-5′-FAM- | | 3 | |
| | GCTAATGGGACGCATGCCTATCTTACAGCT- | | | |
| | TAMRA-3′ | | | |
| Tf | Tf-F 5′-ATCCTGGCTCAGGATGAACG-3′ | 226 | 4 | 16S |
| | Tf-R 5′-TACGCATACCCATCCGCAA-3′ | | 5 | rRNA |
| | Fluorescent probe | | | |
| | Tf-5′-FAM- | | 6 | |
| | ATGTAACCTGCCCGCAACAGAGGGATAAC- | | | |
| | TAMRA-3′ | | | |
| Td | Td-F 5′-AGAGCAAGCTCTCCCTTACCGT-3′ | 105 | 7 | 16S |
| | Tf-R 5′-TAAGGGCGGCTTGAAATAATGA-3′ | | 8 | rRNA |
| | Fluorescent probe | | | |
| | Td-5′-FAM- | | 9 | |
| | CAGCGTTCGTTCTGAGCCAGGATCA- | | | |
| | TAMRA-3′ | | | |
| Pi | Pi-F 5′-TGTCGGTTTACTGGCTATGTTCTC-3′ | 117 | 10 | phoC |
| | Pi-R 5′-CTTGTCTGTTGGCCATCTTGAAG-3′ | | 11 | |
| | Fluorescent probe | | | |
| | Pi-5′-FAM- | | 12 | |
| | TCAAAGACGCACGTACCAATCCAGACC- | | | |
| | TAMRA-3′ | | | |
| Fn | Fn-F 5′-CGCAGAAGGTGAAAGTCCTGTAT-3′ | 101 | 13 | 16S |
| | Fn-R 5′-TGGTCCTCACTGATTCACACAGA-3′ | | 14 | rRNA |
| | Fluorescent probe | | | |
| | Fn-5′-FAM- | | 15 | |
| | ACTTTGCTCCCAAGTAACATGGAACACGAG- | | | |
| | TAMRA-3′ | | | |
| Aa | Aa-F 5′-CAGCATCTGCGATCCCTGTA-3′ | 147 | 16 | lktA |
| | Aa-R 5′-TCAGCCCTTTGTCTTTCCTAGGT-3′ | | 17 | |
| | Fluorescent probe | | | |
| | Aa-5′-FAM- | | 18 | |
| | TCGAGTATTCCTCAAGCATTCTCGCACG- | | | |
| | TAMRA-3′ | | | |

Dental caries (tooth decay) detection service platform: the detected tooth decay-related bacterial strains were as follows:

TABLE 2
Design of Real-time PCR primers and fluorescent probes for tooth
decay-related bacteria

| | | Amplicon | Target | SEQ ID |
|---|---|---|---|---|
| Culture | Primer sequence | size (bp) | gene | NO |
| Sm | Sm-F 5′-GCCTACAGCTCAGAGATGCTATTCT-3′ | 114 | gtfB | 19 |
| | Sm-R 5′-GCCATACACCACTCATGAATTGA-3′ | | | 20 |
| | Fluorescent probe | | | |
| | Sm-5′-FAM- | | | 21 |
| | TGGAAATGACGGTCGCCGTTATGAA-TAMRA- | | | |
| | 3′ | | | |
| Ss | Ss-F 5′-TTCAAAGCCAAGACCAAGCTAGT-3′ | 88 | gtfT | 22 |
| | Ss-R 5′-CCAGCCTGAGATTCAGCTTGT-3′ | | | 23 |
| | Fluorescent probe | | | |
| | Ss-5′-FAM-CCTGCTCCAGCGACAAAGGCAGC- | | | 24 |
| | TAMRA-3′ | | | |

1.2 Collection and Culture of Target Bacteria for Detection of Periodontosis

Target bacteria used in the present invention for detection of periodontosis were purchased from American Type Culture Collection (ATCC) or Bioresource Collection and Research Center of the Food Industry Research and Development Institute in Taiwan. The bacterial strains were as follows: (1) Pg: Porphyromonas gingivalis; (2) Tf: Tannerella forsythia); (3) Td: Treponema denticola; (4) Pi: Prevotella intermedia; (5) Fn: Fusobacterium nucleatum; and (6) Aa: Actinobacillus actinomycetemcomitan. The culture method and condition are summarized in Table 3.

TABLE 3
Periodontosis-related bacterial strains and culture
methods

| | | | | Culture |
|---|---|---|---|---|
| Bacterium | Strain No. | Culture medium | Temperature | environment |
| Porphyromonas | BCRC 14417, | 1. BCRC medium 72, | 37° C. | 1. |
| gingivalis | | TRYPTIC SOY AGAR | | Anaerobic |
| | | (TSA) with 5% Defibrinated | | 2. |
| | | Sheep Blood | | Anaerobic |
| | | 2. TSB with 0.5% yeast | | gas mixture, |
| | | extract,) \acute{ } 0.05% cysteine | | 10% H₂, 5% |
| | | HCl-H₂O, 0.5 mg/ml hemin) | | CO₂ and |
| | | and 2 μg/ml vitamin K1 [33] | | 85% N₂ |
| Tannerella | ATCC 33277 | 1. ATCC ® Medium 1928: | 37° C. | 1. |
| forsythia | | PY Medium (ATCC medium | | Anaerobic |
| | | 1524) with horse serum and | | gas mixture, |
| | | NAM) | | 80% N₂, |
| | | ATCC ® Medium 1921: | | 10% H₂, |
| | | NAM medium | | 10% CO₂ |
| | | 2. TSB with 0.5% yeast | | 2. |
| | | extract, 0.05% L-cysteine | | Anaerobic |
| | | HCl-H₂O, 0.5 mg/ml hemin | | gas mixture, |
| | | and 2 μg/ml vitamin K1 | | 10% H₂, 5% |
| | | | | CO₂ and |
| | | | | 85% N₂ |
| Treponema | ATCC33521 | 1. ATCC ® Medium 1494: | 37° C. | 1. |
| denticola | | Modified NOS medium, | | Anaerobic |
| | | ATCC ® Medium 260: | | gas mixture, |
| | | Trypticase soy agar/broth | | 80% N₂, |
| | | with defibrinated sheep | | 10% CO₂, |
| | | blood | | 10% H₂ |
| | | 2. TSB with 0.5% yeast | | 2. |
| | | extract, 0.05% L-cysteine | | Anaerobic |
| | | HCl-H₂O, 0.5 mg/ml hemin | | gas mixture, |
| | | and 2 μg/ml vitamin K1 | | 10% H₂, 5% |
| | | | | CO₂ and |
| | | | | 85% N₂ |
| Prevotella | BCRC 14477, | 1. BCRC medium 72 | 37° C. | 2. |
| intermedia | ATCC 25611 | TSA with 5% Defibrinated | | Anaerobic |
| | | Sheep Blood | | gas mixture, |
| | | 2. TSB with 0.5% yeast | | 10% H₂, 5% |
| | | extract, 0.05% cysteine HCl- | | CO₂ and |
| | | H₂O, 0.5 mg/ml hemin and 2 μg/ml | | 85% N₂ |
| | | vitamin K1 | | |
| Fusobacterium | BCRC10681, | 1. BCRC medium 53 | 37° C. | Anaerobic |
| nucleatum | ATCC 25586 | REINFORCED | | gas mixture, |
| | | CLOSTRIDIAL MEDIUM | | 10% H₂, 5% |
| | | (OXOID CM149) | | CO₂ and |
| | | ‘Lab-lemco’ powder 10.0 g | | 85% N₂) |
| | | Peptone 10.0 g, | | |
| | | Cysteine•HCl 0.5 g, NaCl | | |
| | | 5.0 g, CH3COONa 3.0 g, | | |
| | | Soluble starch 1.0 g, Glucose | | |
| | | 5.0 g, Yeast extract 3.0 g, | | |
| | | Agar 0.5 g | | |
| | | Distilled water 1.0 L | | |
| | | The pH value is adjusted to | | |
| | | 6.8, boiled to dissolve | | |
| | | completely and autoclaved at | | |
| | | 121° C. for 15 min. | | |
| | | 2. TSB with 0.5% yeast | | |
| | | extract, 0.05% L-cysteine | | |
| | | HCl-H₂O, 0.5 mg/ml hemin | | |
| | | and 2 μg/ml vitamin K1. | | |
| Actinobacillus | BCRC 14189, | 1. BCRC medium 72 | 37° C. | 1. 5% CO₂ |
| actinomycete | ATCC 29523 | TSA with 5% Defibrinated | | 2. |
| mcomitans | | Sheep Blood | | Anaerobic |
| | | 2. TSB with 0.6% yeast | | gas mixture, |
| | | extract, 5% horse serum, 75 μg/ml | | 10% H₂, 5% |
| | | bacitracin and 5 μg/ml | | CO₂ and |
| | | vancomycin | | 85% N₂ |

1.3 Collection and Cultivation of Non-Target Bacteria

Large amounts of bacteria were contained in the oral cavity, through metagenomic analysis, roughly thirty bacteria accounted for 80-97.6% of oral bacteria were identified. To assure the accuracy of the bacterial detection and free of false positive results, other bacteria existing in the oral cavity in large amount were selected as the negative control. The selected strains were Capnocytophaga, Corynebacterium, Veillonella, Neisseria, Leptotrichia, Selenomonas, Rothia, Campylobacter, Eubacterium, etc. As to the negative control, in addition to the non-target bacteria, DNA of other target bacteria were also used as the negative control group for the detection of a specific bacterium.

1.4 Extraction of Bacterial Genomic DNA

After the bacterial culture was completed, an inoculation loop was used to scrape bacteria off and the genomic DNA was extracted according to the standard genomic DNA extraction procedure.

1.6 Establishment of the Real-Time PCR Standard Method.

The method of present invention mainly used specific primers and specific probes (as shown in Table 1) to detect DNA fragments of specific microbes in the oral cavity, respectively. A fluorescent probe (TaqMan probe) was used to detect the product of each PCR reaction to determine quantitatively the concentration of bacterial DNA, and then to estimate the amount of a specific bacterium in the oral cavity. Therefore, a standard curve was established by subjecting the DNA extracted from a standard periodontal bacterial strain to serial dilution and followed by real time PCR, and then the amount of the bacterium was determined by estimating the total weight according to the molecular weight of the genomic DNA of the bacterium.

The following is the estimated standard curve of each periodontal microbial strain and the estimated amount of each bacterium.

(1) Porphyromonas gingivalis; Pg

The formula for the standard curve was: Concentration=10̂(−0.303*C+9.660)

TABLE 4
Calculated values established by using the Pg
standard curve

| Pg Standard | Threshold: 0.03 | | |
|---|---|---|---|
| Curve | Total Weight | | |
| Pg (pg/μl) | (pg) | Bacteria count | CT Value |
| 20000 | 500000 | 2.00E+08 | 18.18 |
| 2000 | 50000 | 2.00E+07 | 21.32 |
| 200 | 5000 | 2.00E+06 | 23.7 |
| 20 | 500 | 2.00E+05 | 26.88 |
| 2 | 50 | 2.00E+04 | 31.55 |
| 0.2 | 5 | 2.00E+03 | 33.95 |
| 0.02 | 0.5 | 2.00E+02 | 36.88 |
| 0.002 | 0.05 | 2.00E+01 | 42.15 |
| 0.0002 | 0.005 | 2.00E+00 | 43.77 |

R\hat{ }2 Value: 0.99392;
Sensitivity: 0.0002 pg/μl

(2) Tannerella forsythia; Tf

The formula for the standard curve was: Concentration=10̂(−0.255*CT+8.404).

TABLE 5
Calculated values established by using the Tf
standard curve

| Tf IC Standard | | | |
|---|---|---|---|
| Curve | Threshold: 0.05 | | |
| Tf (pg/μl) | Total Weight (pg) | Bacteria count | CT Value |
| 6512 | 162800 | 4.40E+07 | 18.64 |
| 651.2 | 16280 | 4.40E+06 | 21.44 |
| 65.12 | 1628 | 4.40E+05 | 25.74 |
| 6.512 | 162.8 | 4.40E+04 | 30.32 |
| 0.6512 | 16.28 | 4.40E+03 | 32.76 |
| 0.06512 | 1.628 | 4.40E+02 | 37.61 |
| 0.006512 | 0.1628 | 4.40E+01 | 42.18 |
| 0.0006512 | 0.01628 | 4.40E+00 | |

R\hat{ }Value: 0.99488;
Sensitivity: 0.006512 pg/μl

(3) Prevotella intermedia; Pi

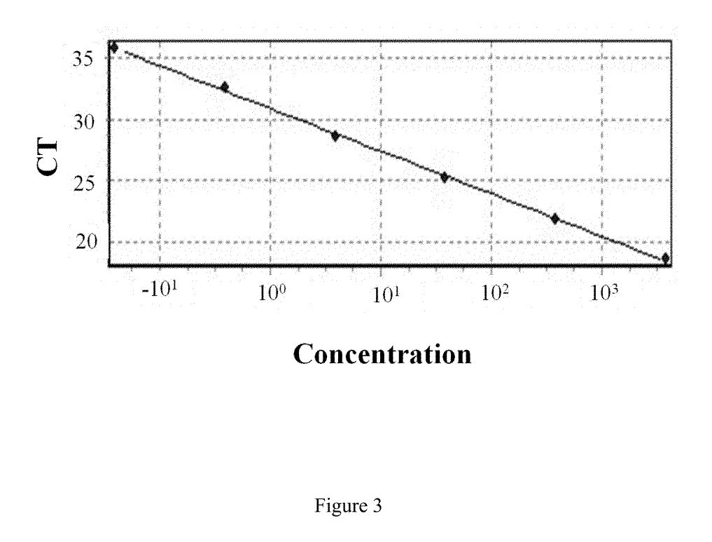

The formula for the standard curve was: Concentration=10̂(−0.290*CT+8.950). Table 6 and FIG. 3 list the calculated values established by using the Pi standard curve.

TABLE 6
Calculated value established by using the Pi
standard curve

| Pi Standard Curve | Threshold: 0.05 | | |
|---|---|---|---|
| Pi (pg/μl) | Total Weight (pg) | Bacteria count | CT Value |
| 3862 | 96550 | 4.20E+07 | 18.66 |
| 386.2 | 9655 | 4.20E+06 | 21.87 |
| 38.62 | 965.5 | 4.20E+05 | 25.26 |
| 3.862 | 96.55 | 4.20E+04 | 28.62 |
| 0.3862 | 9.655 | 4.20E+03 | 32.51 |
| 0.03862 | 0.9655 | 4.20E+02 | 35.75 |
| 0.003862 | 0.09655 | 4.20E+01 | |
| 0.0003862 | 0.009655 | 4.20E+00 | |

R\hat{ }2 Value: 0.99929;
Sensitivity: 0.03862 pg/μl

(4) Fusobacterium nucleatum; Fn

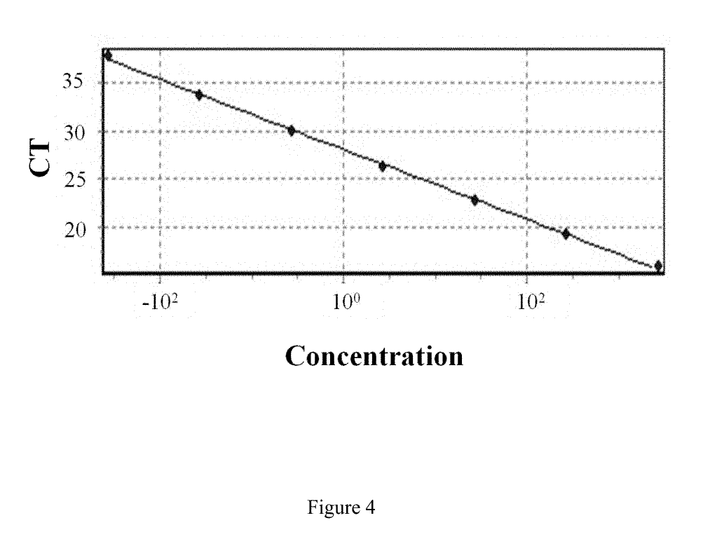

The formula for the standard curve was: Concentration=10̂(−0.385*CT+9.452). Table 7 and FIG. 4 list the calculated values established by using the Fn standard curve.

| Standard Curve | Threshold: 0.05 | | |
|---|---|---|---|
| Fn (pg/μl) | Total Weight (pg) | Bacteria count | CT Value |
| 20000 | 500000 | 2.08E+08 | 12.81 |
| 2000 | 50000 | 2.08E+07 | 15.93 |
| 200 | 5000 | 2.08E+06 | 19.03 |
| 20 | 500 | 2.08E+05 | 22.09 |
| 2 | 50 | 2.08E+04 | 24.08 |
| 0.02 | 0.5 | 2.08E+02 | 27.35 |
| 0.002 | 0.05 | 2.08E+01 | 30.63 |
| 0.0002 | 0.005 | 2.08E+00 | 35.49 |

R\hat{ }2 Value: 0.99909;
Sensitivity: 0.0002

(5) Actinobacillus actinomycetetemcomitans; Aa

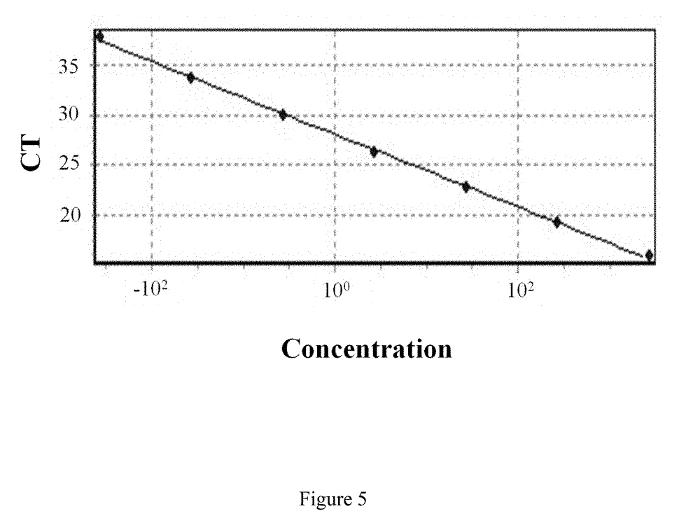

The formula for the standard curve was: Concentration=10̂(−0.274*CT+7.703). Table 8 and FIG. 5 list the calculated values established by using the Aa standard curve

| Aa Standard Curve | | | |
|---|---|---|---|
| Aa (pg/μl) | Total Weight (pg) | Bacteria count | CT Value |
| 2800 | 70000 | 3.04E+07 | 15.79 |
| 280 | 7000 | 3.04E+06 | 19.19 |
| 28 | 700 | 3.04E+05 | 22.61 |
| 2.8 | 70 | 3.04E+04 | 26.22 |
| 0.28 | 7 | 3.04E+03 | 29.98 |
| 0.028 | 0.7 | 3.04E+02 | 33.59 |
| 0.0028 | 0.07 | 3.04E+01 | 37.75 |
| 0.00028 | 0.007 | 3.04E+00 | |

R\hat{ }2 Value: 0.99954;
Sensitivity: 0.0028

Therefore, the molecular weight was used to estimate the number of a bacterium. Using Pg as an example, the calculation was as follow:

by=650 Da
molecular weight of Pg was 2.35 Mb; 2.35 Mb=1.53×10⁹ Da 1.53×10⁹ Da/6×10²³=2.54×10⁻¹⁵ g=2.5 fg=0.0025 pg
→The weight of one Pg bacterium was 2.5 fg, and the standard curve established from Ct values was used to estimate the number of the bacterium in the sample.

The sensitivity obtained from the Pg standard curve was 0.0002 pg/μl. 5 μl was required for the reaction, which was 0.0002 pg×5=1 fg, meaning that one Pg could be detected at this sensitivity level. Using the standard curve for estimation, the quantitative linear range for the number of Pg was 1×10¹˜1×10⁸.

1.8 Molecular Detection of Oral Periodontal Microflora

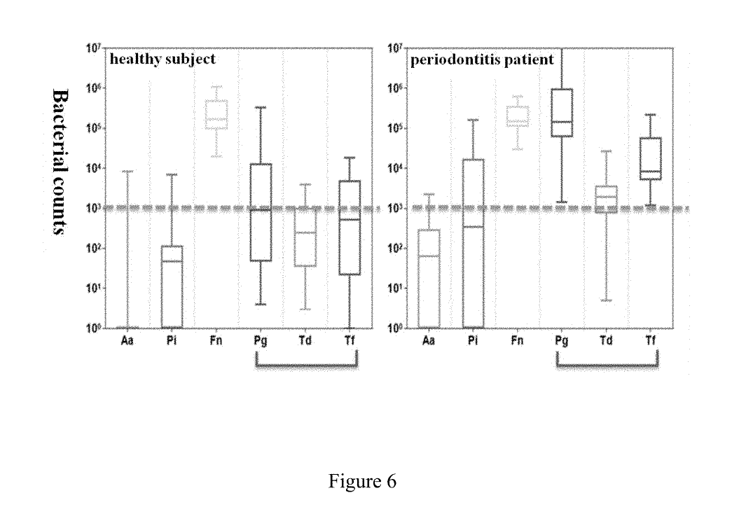

Molecular detections were performed on 20 healthy subjects and 20 patients suffering from periodontosis, and the results of these two groups were analyzed and compared (as shown in FIG. 6). With respect to the 20 healthy subjects, their average age was less than 30 years old, 6 types of oral periodontal bacteria were analyzed, Pg, Td and Tf, three types of bacteria which caused severe periodontosis, were all red complex bacteria. When an average value was used to define a standard, these three varieties of bacteria were defined as 1000, 600, and 800, respectively.

Aa, Pi, and Fn were the other three varieties of bacteria which facilitated the formation of periodontosis, wherein Aa showed significant individual differences. No Aa was found in 15 tested subjects, showing that this bacterium was related with aggressive periodontitis and coronary heart disease and requiring only small amount of bacteria to cause the disease. As long as this bacterium existed, there would be a high chance of severe periodontosis. Thus, the standard value for this bacterium was defined as 100. Pi was defined as 100 based on an average value of healthy subjects. Furthermore, there was a large amount of Fn in the oral cavity, which played an important role in the early and final developmental stages of periodontosis, which easily caused plaque accumulation. Fn was considerably related to daily oral care and the standard value was defined as 10000.

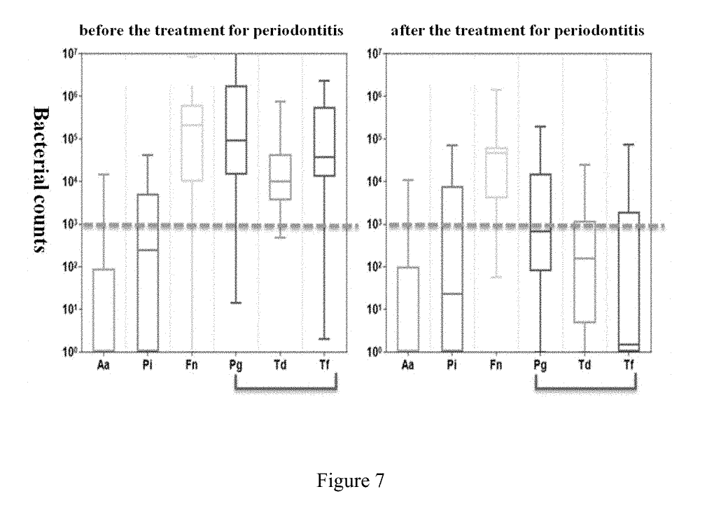

Furthermore, according to the results of the molecular detection of 20 periodontosis patients before and after the treatment of periodontosis, three varieties of bacteria causing severe periodontosis were Pg, Td, and Tf. The average values of Pg, Td and Tf before the treatment were 100000, 10000, and 50000, respectively and decreased to below the standard value after the treatment (as shown in FIG. 7). Real-time PCR molecular assays were used to establish a set of most optimal individualized medical indicators, which became effective bases for daily oral care, diagnosis and course of treatment of the periodontosis, antibiotic treatment for patients suffering from severe periodontosis, as well as follow-up and investigation needed by patients after having dental implants.

Furthermore, to determine dental caries: the standard value for the amount of Streptococcus mutans was 300 to 3000; and the standard value for the amount of Streptococcus sobrinus was 300 to 3000.

2. Establishing Oral Microflora Database

Samples of periodontosis were collected also for establishing an oral microfloral database. In this invention, the MALDI Biotyper technology was used to identify a variety of bacterial strains in the oral cavity. After microorganisms in the samples were cultured, a single bacterial strain was selected, and then the mass spectrometer was used to obtain a protein profile of the bacterial strain. The protein profile, combined with the MSP (main spectra profile) database established by Bruker, was able to identify those microorganisms having high expression level of 16S ribosomal protein. After being compared with the database, the information of the bacterial strain was quickly obtained. New oral bacterial strains and new oral ecological system could be discovered by using this microfloral analysis technique or possibly by using this method.

2.1 Establishing Method of the MALDI Biotyper Technique

Standardized anaerobic culture process was established by using standard periodontal disease bacterial strains, and then the MALDI Biotyper technique was used to identify the bacterial strains of those periodontal bacteria readily available for cultivation. The detailed process is as follow:

(1) Establishing Standarized Anaerobic Culture Conditions

An anaerobic microbial culture environment was established, different oxygen content status as well as culture media were designed, and then subgingival or saliva samples were inoculated onto suitable culture media.

(2) Bacterial Culture of Clinical Samples

A variety of culture media matching with different oxygen concentrations were used to culture multiple combinations of microorganisms in order to collect the most diverse varieties of microorganisms, and to establish a rich database of oral microorganisms.

(3) Establishing an Oral Microflora Database

After the bacterial culture of clinical samples were completed, a sample of gingiva or saliva was inoculated onto a suitable culture medium, and cultured for 18 or 24 hours. Then a micropipette was used to collect colonies, smeared the collected colonies onto a clean MALDI sample plate, then added 1 μl of 70% formic acid into the MALDI sample plate, after air-dried under room temperature, 1 μl of HCCA matrix solution was added, air-dried under room temperature again, then placed the sample plate on the MALDI-TOF mass spectrometer for analysis and identification of the bacteria. Finally, the spectra generated by the MALDI-TOF mass spectrometer were compared with the database MSP (main spectra profile) established by Bruker corporation. The MSP database contained 5,627 standard bacterial strains, the results of microbiological identification were obtained as a result of comparison.

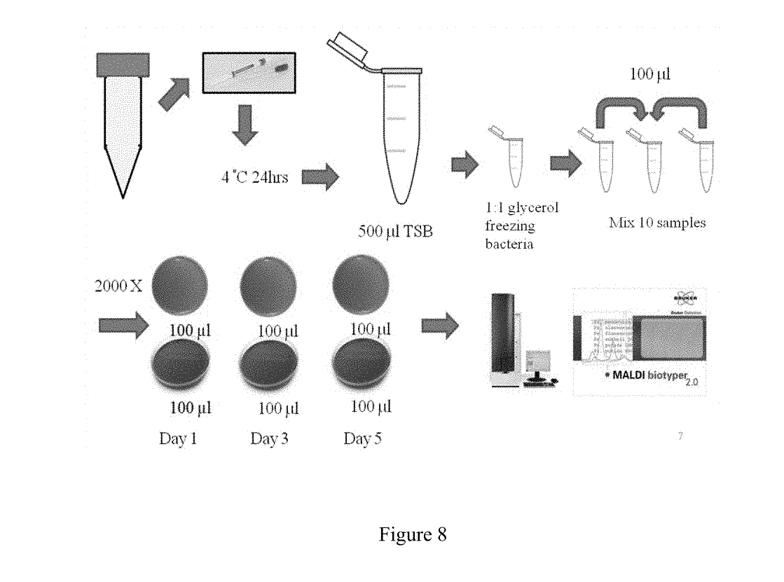

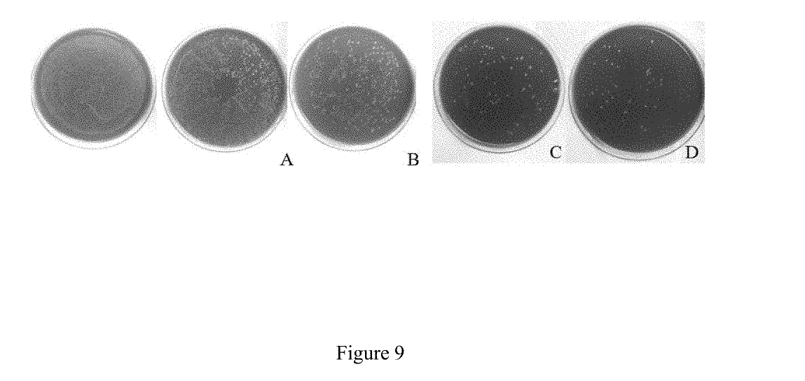

Therefore, the present invention utilized saliva collected from the tested subjects as samples to confirm the conditions for culturing oral periodontal bacteria. FIG. 8 shows the establishment of the handling method used before the sample identification procedure was performed by the MALDI Biotyper. The clinical sample delivery process was simulated. First, samples collected from volunteers were stored in an anaerobic delivery device, placed at 4° C. for 24 hours, then liquid culture medium (for example, trypsin soy broth (TSB)) was used to dissolve the samples, and then a freeze-tube test was carried out. To meet the requirements of MALDI Biotyper, after the samples were diluted, different anaerobic media (at least sheep blood culture medium and chocolate culture medium) were used in order to obtain a large amount of highly divergent oral bacteria (as shown in FIG. 9); and finally, the MALDI Biotyper was used for identification.

The present invention was directed to healthy subjects and periodontosis patients, samples were collected from 10 subjects of each group. The collected samples were mixed and then cultured with different anaerobic culture media to obtain a large amount of highly divergent oral bacteria. Colonies of single variety of microorganism was cultured under an anaerobic environment, more than 2000 colonies of single variety of microorganism were selected from each of the tested groups for identification.

The MALDI Biotyper technique was used to identify the bacterial strains presented in the oral cavity. A protein profile was generated by a mass spectrometer and then the MSP database of MALDI Biotyper (Bruker Biotyper) was used for comparing to identify those microorganisms having a high expression level of 16S ribosomal protein. Then through comparison with the database, the information required for bacterial strain identification was obtained to analyze the oral microflora.

2.3 Distribution of Bacterial Strains Causing Severe Periodontitis in Healthly Subjects and Periodontitis Patients

The target bacterial strains that caused periodontosis were Actinobacillus actinongcetenicomitans, Fusobacterium nucleatuni and Porphyromonas gingivalis, which only accounted for 0˜0.1% of the bacteria in the saliva and subgingival samples collected from healthy subjects; and 0%-0.1% of the bacteria in the saliva samples collected from the periodontosis patients.

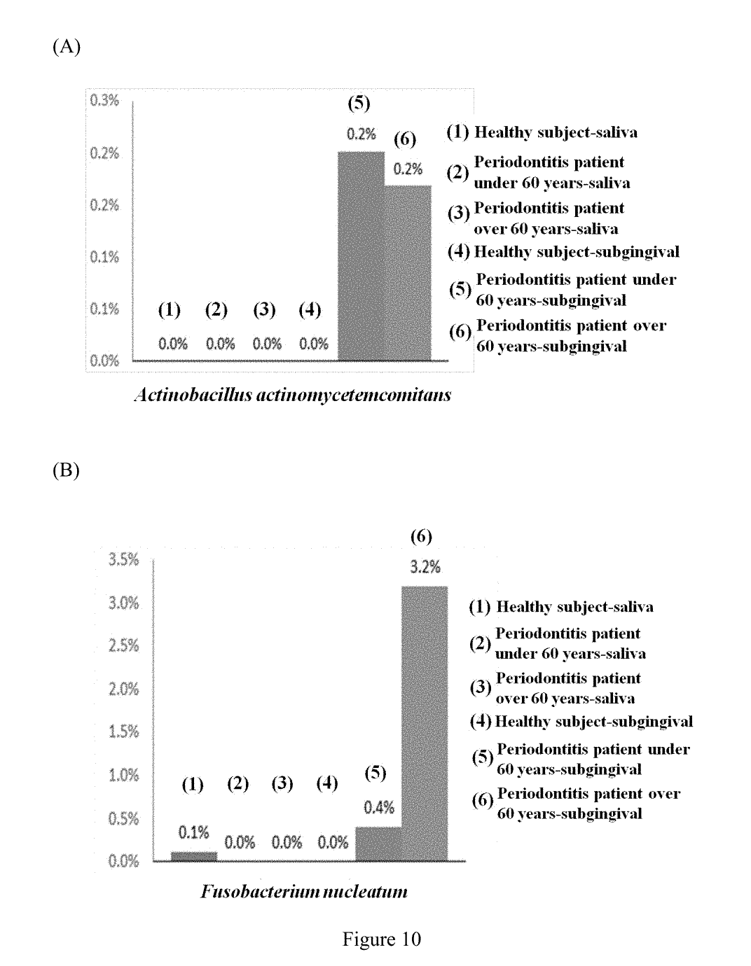

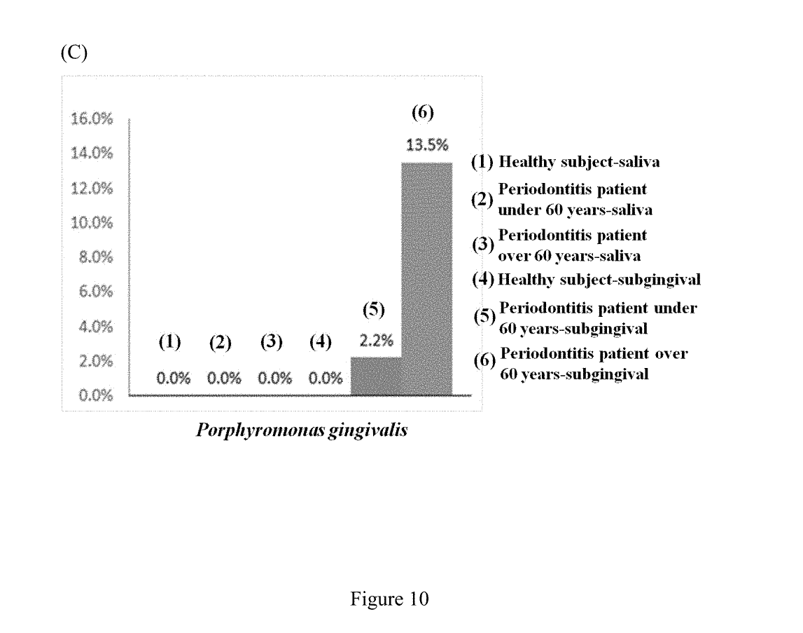

However, the amount of target bacterial strains in the subgingival samples collected from the periodontosis patients was significantly higher. Actinobacillus actinongcetenicomitans accounted for 0.15˜2% in the subgingival samples collected from patients with periodontosis. The other two varieties of bacteria, Fusobacterium nucleatuni and Porphyromonas gingivalis, showed age-dependent difference. Fusobacterium nucleatuni accounted for 0.4% in the periodontosis patients under the age of 60, but accounted for 3.2% in the elderly over the age of 60. Porphyromonas gingivalis, which caused severe periodontosis, showed even more significant difference. Porphyromonas gingivalis accounted for 2.2% in the periodontosis patients under the age of 60, but accounted for 13.5% in the elderly over the age of 60. A comprehensive analysis of the oral microflora conducted by using the MALDI Biotyper was able to define the indicators for severe periodontosis (as shown in FIGS. 10A-10C).

The MALDI Biotyper system was used to identify oral microorganisms in healthy subjects, periodontosis patients, and patients with dental implants, to analyze and to establish a database so that even more accurate clinical diagnosis and analysis could be provided in the future.

3. Molecular Detection Conducted Before and After the Use of Antibacterial Mouthwash

The present invention collected oral saliva samples from six healthy subjects before and after continuous and intensive use of an antimicrobial peptide mouthwash for three days, four times a day. The purpose was to evaluate the differences before and after the use of the antimicrobial peptide mouthwash and to analyze the efficacy thereof. The antimicrobial peptide mouthwash comprised a peptide from the histatin family and its derivatives as components, wherein the peptide from the histatin family comprised a fragment of P-113 peptide fragment consisting of 12 amino acids, and antifungal and antibacterial peptide derivatives thereof. The peptide sequence (SEQ ID NO: 25) may be referred to the U.S. Pat. Nos. 5,486,503, 5,631,228, 5,646,119, 5,696,078, 5,885,965 and 5,912,230.

The present method utilized a highly sensitive and highly specific real-time PCR to conduct experiments. By using a thermal cycling step, small amount of DNA was amplified to accomplish the goal of amplification. During the reaction, a DNA binding dye or a fluorescent probe was added, the amount of fluorescence generated in each cycle was detected. As PCR products increased with each cycle, the fluorescence intensity also increased as the PCR products increased. The real-time PCR recorded the fluorescence intensity in each PCR cycle, through which the results were read and assessed.

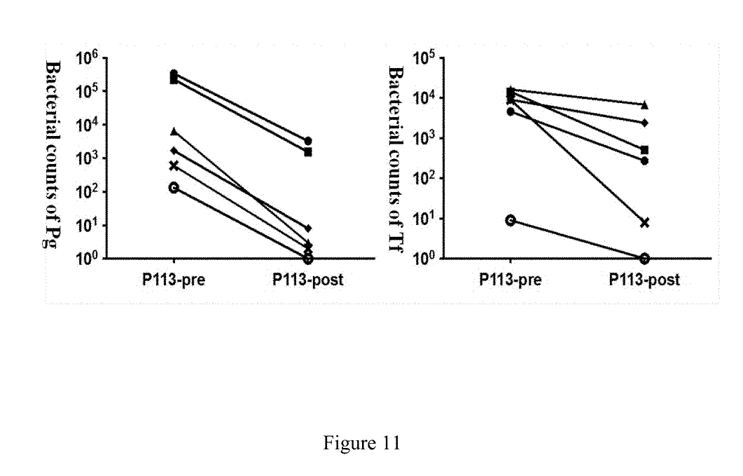

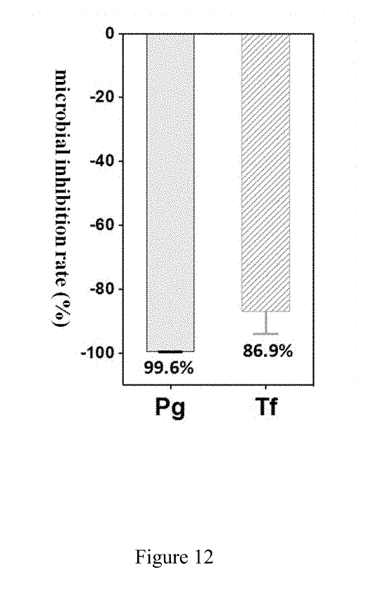

The results of this experiment designed the primers based on the highly specific 16S rRNA gene sequences of Porphyromonas gingivalis and Tannerella forsythia, analyzed the DNAs in the saliva samples and then compared with the amount of standard bacterial strains to effectively quantify the results of the distribution of the amount of oral bacteria. The experimental results showed that the amount of Porphyromonas gingivalis (Pg) in six tested subjects significantly decreased before and after the use of P-113. It also showed that the amount of Tannerella forsythia (Tf) also significantly decreased before and after the use of the mouthwash containing the antimicrobial peptide P-113 (as shown in FIGS. 11A and 11B). Furthermore, the mouthwash containing the P-113 antimicrobial peptide of the present invention was able to significantly inhibit the growth of Porphyromonas gingivalis and Fusarium moniliforme (as shown in FIG. 12).

In addition, the present invention also studied the effect of the mouthwash containing P-113 antimicrobial peptide on other oral colonies. Fifteen healthy subjects continuously used the P-113 mouthwash for three days, four times a day, 1 ml of saliva sample was collected before and after the use of the P-113 mouthwash, the saliva samples of these fifteen subjects were mixed, and then broadly cultured in various different culture media. Among the single-microorgamism colonies cultured under an anaerobic environment, more than 500 single colonies were selected for identification. The MALDI Biotyper technique was then used to identify different varieties of bacteria presented in the oral cavity. The mass spectrometer was used to obtain protein profiles of the bacterial strains. Then the MSP database of the MALDI Biotyper (Bruker Biotyper) was used for comparison to identify those microorganisms with high expression level of 16S ribosomal protein. After comparisons were made with the database, the information of the bacterial strains were quickly obtained, and the oral microflora were analyzed with this technique.

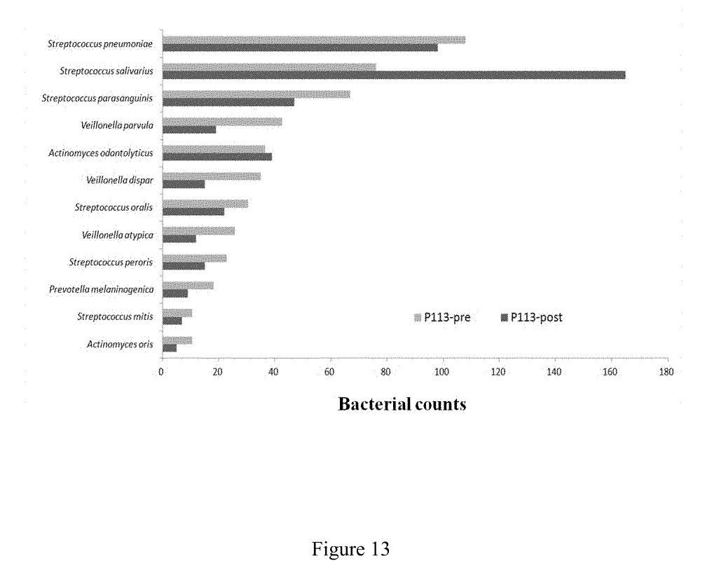

The mixed samples of fifteen healthy subjects were used for culturing, the amounts of bacteria were identified before and after the use of P-113 mouthwash for comparison. When the identified bacteria were ranked from high to low, it was found that after the use of the P-113 mouthwash, the amounts of most bacteria tended to decrease. Streptococcus pneumonia was one of the dominant bacteria, a spherical and gram-positive bacterium with α-hemolysin, an important human pathogenic bacterium that could cause pneumonia; Streptococcus parasanguinis was one of the early formation bacteria for dental plaque, when invaded into bloodstream, Streptococcus parasanguinis would attach to the damaged or prosthetic valve to cause opportunistic endocartitis; the amounts of both bacteria decreased significantly. A very important discovery was that Streptococcus salivarius significantly increased by more than 2-fold. It was a probiotic bacterium, which had a great effect on the maintenance of the oral pH, tooth decay prevention, and the maintenance of the ecological balance of oral microflora. Furthermore, the amount of Streptococcus salivarius was relatively small in those who had bad breath, and had inhibitory effect on bacteria related to bad breath (as shown in FIG. 13)

Therefore, through evaluations conducted before and after the periodontitis treatment procedure, and the use of antimicrobial mouthwash (such as the mouthwash containing antimicrobial peptide P-113) whether the oral microflora could be effectively balanced was determined. Evaluation was carried out with the oral microflora molecular detection platform to determine the efficacy of the course of treatment, which was also the basis for establishing the oral care platform for follow-up of periodontosis treatment subsequent to the microflora molecular detection.

4. Oral Microflora Detection System

The present invention established an oral microflora detection system for follow-up of the periodontal condition of periodontosis patients. After the patients received a course of treatment for periodontosis and oral care, the system was able to monitor the efficacy effectively and established personalized oral health indicators. Thus, after the molecular detection was conducted for monitoring periodontosis, each periodontosis patient received a detection report to understand his or her own periodontal condition. The report also served as a basis for future self oral management and oral care.

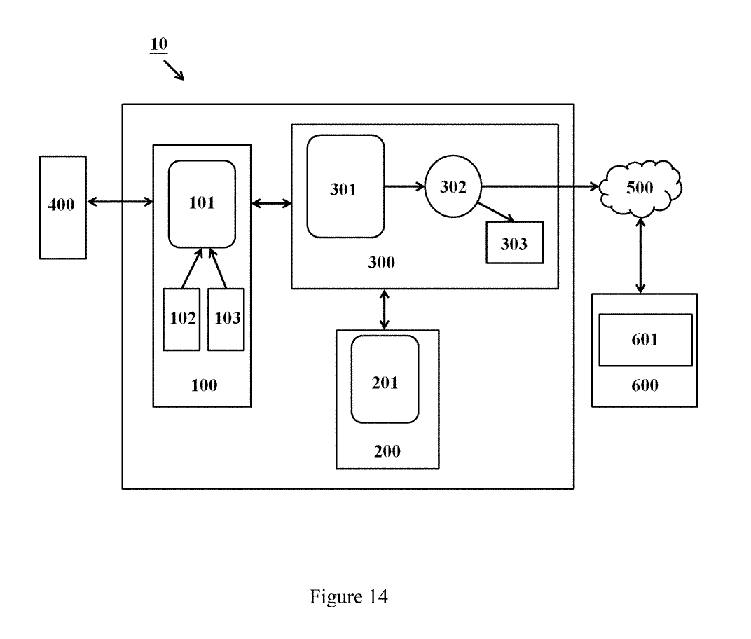

As shown in FIG. 14, the present invention provided an oral health detection and evaluation system 10 comprising: (1) an oral microflora detecting device 100 which was used for detecting a distribution of the oral microflora in an oral sample and for generating a microflora distribution data 101; (2) an oral microflora colony database 200 which stored an oral microflora colony comparison table 201; and (3) an oral health evaluating device 300 connected to the oral microflora detecting device 100 and the oral microflora colony database 200, which was used for comparing the microflora distribution data 101 and the oral microflora colony comparison table 201 and for generating an oral health evaluating table 301 based on the comparison results.

The oral health detection and evaluation system 10 further comprised a sampling device 400 connected to the oral microflora detecting device 100, which was used for collecting the oral sample. The oral sample was an oral mucosa, a subgingival mucosa or a saliva. Therefore, through the sampling device 400, an oral sample was collected from a to-be-tested subject, and through the oral microflora detecting device 100, the microbes in the oral sample were analyzed and detected.

The oral microflora detecting device 100 comprised a PCR device 102 and a microflora analyzer 103. The PCR device 102 used a specific primer of a specific microbe (such as a periodontosis-related bacterium) to perform polymerase chain reaction of DNA in the oral sample, amplified the quantity of the DNA to determine quantitatively the concentration of the tested microbe and then to estimate the amount of a specific microbe. The microflora analyzer 103 was a MALDI-TOF mass spectrometer which could be used to analyze and identify microbes. Thus, the detection and analysis results obtained by the PCR device 102 and the microflora analyzer 103 were used to establish the microflora distribution data 101, and the microflora distribution data 101 therefore comprised a set of data for identifying the varieties of microbes and a set of data for the amounts of microflora colonies. The varieties of microbes referred to periodontosis-related indicator microbes, which included but not limited to: Porphyromonas gingivalis; Tannerella forsythia; Treponema denticola; Prevotella intermedia; Fusobacterium nucleatuni; and Actinobacillus actinomycetenicomitans. Furthermore, the varieties of microbes referred to dental caries-related indicator microbes, which included but not limited to: Streptococcus mutans and Streptococcus sobrinus.

The oral health detection and evaluation system 10 could be used to diagnose a risk of suffering from periodontosis or dental caries. Thus, the oral microflora colony database 200 comprised an oral health control group data, a microbial characteristic data, a periodontosis-related data, and a dental caries-related data. The oral health control group data comprised a set of data for the distributions and the amounts of microbes in a healthy oral cavity, which was used for comparing with and analyzing microbial colonies in a to-be-tested oral sample. The microbial characteristic data comprised a set of data of the characteristics of a variety of oral microbes and periodontal diseases possibly to be induced. The periodontosis-related data comprised the risk and the progression determination of periodontosis and related symptoms/diseases thereof. The dental caries-related data comprises the risk and the progression determination of dental caries and related symptoms/diseases thereof. The above-mentioned data were stored in the oral microflora colony database, and the oral microflora colony database 200 would be continuously updated and the information would be expanded, in order to provide the most up to date determination criteria for oral health evaluation and periodontosis risk assessment.

Thus, the oral microflora colony database 200 utilized the stored data to generate the oral microflora colony comparison table 201, which comprised a comparison table of the varieties and standard values for the amounts of periodontal disease-related indicator microbes and a comparison table for periodontosis-related diseases. Furthermore, the oral microflora colony comparison table 201 also comprised a comparison table of the varieties and standard values for the amounts of dental caries-related indicator microbes and a comparison table for dental caries-related diseases.

Using the diagnosis of periodontal disease as an example, the oral health evaluating device 300 received the oral microflora distribution data 101 from the oral microflora detecting device 100 and the oral microflora colony comparison table 201 of the oral microflora colony database 200, then analysis and comparison were carried out to determine the differences between the varieties and the amounts of the microbes in the oral sample and the varieties and standard values for the amount of the periodontosis-related indicator microbes, which would generate an index table for comparison of the amounts of periodontal disease-related microbes. Then, a risk for suffering from periodontosis-related disease was further determined according to the identified differences resulting from the comparison; i.e., an evaluating table for periodontal-related disease risk index is therefore generated. Therefore, the oral health evaluating table 301 comprised an index table for comparison of the amounts of periodontosis-related indicator microbes and an evaluating table for periodontal disease-related risk index. In addition, the oral health evaluating table 301 further comprised an index table for comparison of the amount of dental caries-related indicator microbes, and the evaluating table for dental caries-related risk index.

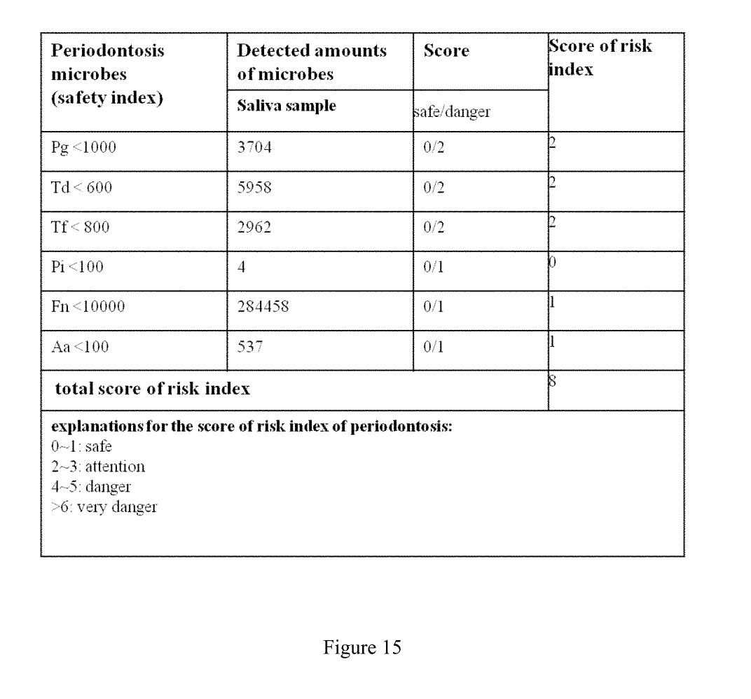

As shown in FIG. 15, the oral health evaluating table utilized the information of the periodontosis risk assessment table as an example, which comprised the amounts of periodontosis-related indicator microbes in the tested sample, the safety index for the amounts of microbes, the score of risk index for each microbe, the total score of risk index, and explanations for the total score. Based on this detection report, it was found that when the amount of Porphyromonas gingivalis (Pg) colonies was more than 1000, the score of risk index was 2; when the amount of Treponema denticola (Td) colonies was more than 600, the score of risk index was also 2. The risk index for periodontitis was evaluated according to the sum of total final scores. The dental caries risk assessment table could be designed by referring to the information contained in the periodontosis risk assessment table.

In addition, the oral health evaluating device 300 further comprised an analysis result storage element 302 which could be used to store the oral health evaluating table 301. The oral health evaluating device 300 further comprised a result output device 303, connected to the analysis result storage element 302, for outputting the oral health evaluating table 301. The result output device 303 was a network transmission device, a telefax facsimile, a display, a printer, a computer system, an electronic application device or a memory storage device.

In addition, the analysis result storage element 302 was further connected to a cloud server 500. The cloud server 500 could be connected to a personal computer device 600 of the subject to be tested. The personal computer device 600 included but not limited to a smartphone, a desktop computer, a notebook computer, a tablet computer and a smart wearable device. Therefore, through the display device of the personal computer device 600, the subject to be tested could connect with the cloud server 500 via a user interface 601 to receive and view the oral health evaluating table 301 of the analysis result storage element 302 in order to understand the periodontal condition of the individual. Furthermore, the user interface 601 further comprised an online commentary service to explain the content of the oral health evaluating table 301 and to provide recommendations for oral care or treatment.

After the subject to be tested was determined to have a high risk for suffering from periodontal disease, or had suffered from periodontal disease based on the oral health evaluating table 301, the subject to be tested could further receive a scheduled course of medical treatment for periodontal disease offered by a medical institution, such as periodical use of a mouthwash containing antimicrobial peptide P-113, which would effectively balance and inhibit pathogenic microflora in the oral cavity to achieve the effect of oral health maintenance. During the course of medical treatment, the oral health detection and evaluation system 10 of the present invention was used to continuously monitor changes of oral miroflora in order to monitor the efficacy of the treatment, and to establish individualized oral health indicators.

Those skilled in the art recognize the foregoing outline as a description of the method for communicating hosted application information. The skilled artisan will recognize that these are illustrative only and that many equivalents are possible.

What is claimed is:

1. A method for evaluating a risk for suffering from periodontal disease, comprising:
(1) providing an oral sample from a subject;
(2) detecting at least two indicator microbes in the oral sample; and
(3)analyzing amounts lithe at least two indicator mirobes;
wherein when each of the amounts of the at least two indicator microbes is higher than a standard value for an amount of a corresponding indicator microbe, it indicates that the subject has a high risk for suffering from periodontal disease.

2. The method of claim 1, wherein the periodontal disease comprises periodontosis and dental caries.

3. The method of claim 2, wherein the periodontosis comprises periodontitis and gingivitis.

4. The method of claim 1, wherein the oral sample is an oral mucosa, a subgingival mucosa or a saliva.

5. The method of claim 1, wherein the at least two indicator microbes comprise Porphyromonas gingivalis, Tannerella forsythia, Treponema denticola, Prevotella intermedia, Fusobacterium nucleatum, and Actinobacillus actinomycetemcomitans.

6. The method of claim 5, wherein the standard values for the amounts of the corresponding indicator microbes comprise the standard value for the amount of the Porphyromonas gingivalis ranging from 300-3000, the standard value for the amount of the Tannerella forsythia ranging from 100-2000, the standard value for the amount of the Treponema denticola ranging from 100-2000, the standard value for the amount of the Prevotella intermedia ranging from 20-1.000, the standard value for the amount of the Fusobacterium nucleatum ranging from 8000-80000, and the standard value for the amount of the Actinobacillus actinomycetemcomitans ranging from. 20-200.

7. The method of claim 1, wherein the at least two indicator microbes comprise Streptococcus mutans and Streptococcus sobrinus.

8. The method of claim 7, wherein the standard values for the amounts of the corresponding indicator microbes comprise the standard value for the amount of the Streptococcus mutans ranging from 300-3000, and the standard value for the amount of the Streptococcus sobrinus ranging from 300-3000.

9. The method of claim 1, wherein the method for detecting the at least two indicator microbes in the oral sample comprises a PCR, a real-time PCR, a multiplex real-time PCR, a digital PCR, a droplet digital PCR (ddPCR) and a PCR array.

10. The method of claim 1, wherein the method for detecting the at least two indicator microbes in the oral sample comprises MALDI Biotyper detecting method.
